(19) United States
(12) Patent Application Publication
(10) Pub. No.: US 2005/0169866 A1
(43) Pub. Date: Aug. 4, 2005

(54) Emulsion-form hair treatment compositions containing solid particles

(76) Inventors: Manuela Hannich, Egelsbach (DE); Bernd Stein, Hoesbach (DE); Michael Franzke, Rossdorf (DE); Ute Doepner-Reichenbach, Koenigstein (DE); Julia Siefert, Pfungstadt (DE); Iris Haselbauer, Stockstadt (DE)

Correspondence Address: STRIKER, STRIKER & STENBY, 103 EAST NECK ROAD, HUNTINGTON, NY, 11743, US

(21) Appl. No.: 11/045,451

(22) Filed: Jan. 28, 2005

(30) Foreign Application Priority Data
Jan. 29, 2004 (EP) 04001875.6

(51) Int. Cl. A61K 7/06; A61K 7/11

(52) U.S. Cl. 424/70.11

(57) ABSTRACT

The emulsion-form hair treatment composition contains at least 50 percent by weight water; from 5 to 10 percent by weight of at least one hydrophobic substance, preferably a fatty alcohol, vegetable oil, hydrocarbon that is liquid at room temperature and/or silicone oil; at least 15 percent by weight of at least one emulsifier; a hair-fixing polymer, preferably at least one non-ionic and/or cationic vinyl lactam polymer, and an undissolved solid particulate, preferably silica. A method for setting up the hairstyle or hair-do involves applying this hair-treatment composition to dry or wet hair and then putting the hair the hairstyle or hair-do.

### BACKGROUND OF THE INVENTION

1. Field of the Invention

The subject matter of the present invention is an emulsion-form hair treatment composition containing water, a hydrophobic material, an emulsifier, a hair-fixing polymer and an undissolved solid material in particulate form. The invention also concerns a method for setting up a hairstyle or hair-do using the hair treatment composition according to the invention.

2. Related Art

A permanent rasta hair-do is a hairstyle formed by long narrow plaited braids, which usually comprise thin and extremely strongly matted hair strands. This sort of hairstyle is characterized by dreadlocks. Ethnic hair (e.g. African or Afro-american hair, so-called kinky hair) has the ideal prerequisites for setting up a dreadlock and/or rasta hair-do because of its natural properties and natural frizziness or crinkliness. It is also possible to setup a rasta hair-do with other types of hair, among others, also smooth hair, for example central European or Asiatic hair with suitable techniques. However for this purpose it is also necessary to provide a hair structure inclined to be strongly matted prior to setting up the hair-do. This usually occurs by a tedious combination of chemical treatment of the hair with a reducing agent and mechanical treatment (up to several hours of constant back-combing). An intensive irreversible matting of the hair and irreparable damage occurs to the hair. The permanent rasta hair-dos set up with conventional techniques are not removable again without more effort. Many fashion conscious users would like to have a rasta hair-do only in passing or only for a certain occasion, e.g. for a party on the weekend but would like to return to their original hair-do without damage to the hair the next day. Thus there exists a need for a technique to set up a temporary rasta or rasta-like hair-do, which is removable again without hair-damaging treatment steps and in the shortest possible time, which means without tedious back-combining. The so-called “ropes” is a simple variant of the known rasta look. It is a matter of small, twisted hair strands.

### SUMMARY OF THE INVENTION

It is an object of the present invention to provide a hair treatment composition, with which it is advantageously possible to set up a so-called “ropes” hair-do. On the one hand, the hairstyle should have sufficient hold for about a day, especially one to three days. On the other hand the hairstyle should be removable again as easily as possible. The hair treatment composition should be easily distributable portion-wise on the hair strands and should hold together the ropes effectively without being too sticky, without the ropes adhering to each other or the hair feeling unpleasantly sticky. The hair-do should remain elastic after it is set up.

The subject matter of the invention is thus a hair treatment composition, which is in the form of an emulsion comprising an aqueous hydrophilic phase and a hydrophobic phase, wherein said hair treatment composition comprises
(A) at least one 50 percent by weight water, based on a total weight of the composition;
(B) from 5 to 10 percent by weight of at least one hydrophobic substance, based on the total weight of the composition;
(C) at least 15 percent by weight of at least one emulsifier, based on the total weight of the composition;
(D) at least one hair-fixing polymer; and
(E) at least one undissolved solid particulate.
The hair treatment composition according to the invention is characterized by special adhesive properties. It produces sufficient adherence of the individual hairs to each other, which provides the braids or locks with sufficient stability. After drying the hair has a pleasant, not sticky feel. Additional advantages of the product according to the invention include a problem-free washability, preferably only with water without arduous mechanical treatment.

The term “emulsion-form consistency” means a water-in-oil, oil-in-water or microemulsion. The composition according to the invention is preferably a water-in-oil emulsion.

The solvent for the hydrophilic phase can be water or a mixture of water and at least one alcohol. Suitable alcohols are especially monovalent or polyvalent C₁- to C₅-alcohols, such as e.g. ethanol, n-propanol, isopropanol, butanols, pentanols, ethylene glycol, propylene glycols, butylenes glycols, glycerol or pentandiols. Preferred monovalent alcohols are ethanol and isopropanol. Preferred multivalent alcohols are propyleneglycols and glycerol. Water is preferably contained in an amount of from 50 to 65 percent by weight. Alcohols are preferably contained in an amount of 0.1 to 15 percent by weight, especially preferably from 1 to 10 percent by weight.

### Hydrophobic Substance (B)

The hydrophobic substance preferably is contained in the composition according to the invention in an amount of 5 to 10 percent by weight. It can be a solid or liquid at room temperature (25° C.). Suitable hydrophobic substances include, among others, waxes or waxy materials, e.g. natural, after-growth waxes (insect wax, animal wax, vegetable wax), fossil waxes (petroleum wax, brown coal wax, peat wax or ozokerite), synthetic waxes (Fischer-Tropsch wax, Polyethylene waxes or amide wax), high melting paraffins, esters fats, long-chain carboxylic acids or long chain C₁₀- to C₂₂-alcohols, each with melting or solidification points above room temperature (20° C.). Further hydrophobic materials include, especially, oils or oily materials, e.g. naturally occurring after-growing oils (vegetable and animal fat oils), synthetic oils, silicone oils, mineral oils, etheric oils, water-insoluble, branched or linear aliphatic hydrocarbons, linear or branched alcohols, especially liquid fatty alcohols and long-chain ethers or esters, in which the named substances preferably have at least 8 carbon atoms, especially preferably 8 to 22 carbon atoms. Suitable hydrocarbons are, e.g., liquid paraffins, squalane or squalene. Furthermore esters of trivalent or multivalent alcohols, especially vegetable triglycerides, such as olive oils, almond oil, peanut oil, sunflower seed oil and synthetic triglycerides, such as C₈- to C₁₀-trifatty acid glyderide esters, or also jojoba oil. Furthermore mono-esters or diesters of formula R¹—COOR², R¹—COO—R³—OOCR¹, and R²OOC—R³—COOR², wherein R¹ stands for a C₈- to C₂₂-alkyl group, R² stands for a C₃- to C₂₂-alkyl group and R³ stands for a C₂- to C₁₆-alkylene group. Branched primary alcohols, such as those known by the name Guerbeta alcohols, are also suitable. In addition, substances, which are commonly used as turbidity-inducing agents in cosmetic compositions, are suitable for use as hydrophobic substances. These latter substances include especially those of the following formula: R¹—COO—(CHR⁴CHR⁵O)ₙ—COR⁶, wherein R¹ stands for a C₈- to C₂₂-alkyl group, R⁴ and R⁵ stand for hydrogen or methyl and R⁶ for hydrogen or for R¹ and n is a number between 1 and 12, preferably 1, 2, 3 or 4. Glycol difatty acid ester and polyethylene glycol difatty acid ester, which are present at room temperature in solid form, are preferred.

The hydrophobic substance can also be a water-soluble silicone compound, especially silicone oil, e.g. liquid cyclic or linear silicone (dimethyl-polysiloxane), liquid, easily-volatile silicones, cyclic dimethylsiloxanes with 3 to 8, preferably 4 to 6 silicon atoms, especially cyclotetradimethyl siloxane, cyclopentadimethyl siloxane or cyclohexdimethyl siloxane. Additional silicones include dimethylsiloxane/methylalkylsiloxane cyclo-copolymers, e.g. Silicone FZ 3109 of Union Carbide, which is a dimethylsiloxane/methyloctylsiloxane cyclo-copolymer. Suitable silicone oils are, especially polydimethylsiloxanes phenylated silicones, polyphenylmethyl siloxanes, phenyltrimethicone, poly-(C₁-C₂₀)-alkylsiloxanes and alkylmethylsiloxanes.

Fatty alcohols, vegetable oils, hydrocarbons that are liquid at room temperature and silicone oils are especially preferred hydrophobic substances.

### Emulsifier (C)

Preferred embodiments of the emulsion-form hair treatment composition contain from 15 to 30 percent by weight of at least one emulsifier (C). The emulsifiers can be nonionic, anionic, cationic, amphoteric, zwitterionic or mixtures thereof. Suitable emulsifiers are, for example, the emulsifiers described in “International Cosmetic Ingredient Dictionary and Handbook”, 7^{th} Edition, Volume 2, in the “Surfactants” section, especially under the heading “Surfactants—Emulsifying Agents”. Nonionic emulsifiers include, e.g. , ethoxylated fatty alcohols, ethoxylated nonylphenols, fatty acid monoglycerides, fatty acid diglycerides, ethoxylated and hydrogenated or non-hydrogenated castor oil, fatty acid alkanol amides and ethoxylated fatty acid esters. Cationic emulsifers include, e.g., long-chain quaternary ammonium compounds, such as those known under the CTFA name “Quaternium”, such as alkyltrimethyl ammonium salts or dialkyldimethyl ammonium salts with C₈- to C₂₂-alkyl groups. Anionic emulsifiers are, e.g., fatty alcohol sulfates, alkyl ether sulfates and alkylbenzene sulfonates. Amphoteric emulsifiers include, e.g. betaines, such as fatty acid amidoalkylbetaines, sulfobetaines and C₈- to C₂₂-alkylbetaines.

Preferred emulsifiers include, e.g.:
ethoxylated fatty alcohols, fatty acids, fatty acid glycerides or alkylphenols, especially addition products of 2 to 30 mol ethylene oxide and/or 1 to 5 mol propylene oxide with C₈- to C₂₂-fatty alcohols, with C₁₂- to C₂₂-fatty acids or with alkylphenols containing 8 to 15 carbon atoms in their alkyl groups;
C₁₂- to C₂₂-fatty acid monoesters and diesters of addition products of 1 to 30 mol ethylene oxide with glycerol;
addition products of 5 to 60 mol, preferably 20 to 50 mol, of ethylene oxide with castor oil or hydrogenated castor oil; and
monoesters, diesters and/or triesters of phosphoric acid with addition products of 2 to 30 mol of ethylene oxide with C₈- to C₂₂-fatty alcohols;.
fatty acid sugar esters, especially esters of saccharides and one or two C₈- to C₂₂-fatty acids with the INCI names: sugar cocoate, sucrose dilaurate, sucrose distearate, sucrose laurate, sucrose myristate, sucrose oleate, sucrose palmitate, sucrose ricinoleate, sucrose stearate; and
polyglyceryl fatty acid esters, especially of one, two or several C₈- to C₂₂-fatty acids and polyglycerol with preferably 2 to 20 glyceryl units.

A preferred emulsifier mixture comprises nonionic emulsifiers, in which one, two or more emulsifiers are ethoxylated and hydrogenated castor oils and at least one other emulsifier is an ethoxylated fatty alcohol.

### Hair-Fixing Polymer (D)

The hair-fixing polymer (D) is preferably contained in an amount of from 0.1 to 15 percent by weight, especially 0.5 to 10 percent by weight. The hair-fixing polymers can be anionic, cationic, zwitterionic, nonionic and amphoteric polymers or mixtures thereof. They can be natural or synthetic polymers. The term “natural polymer” means a polymer of natural origin, which can be understood as a polymer of natural origin, which also can be subsequently chemically or physically modified. The term “film-forming polymer” means a polymer, which is in a position to deposit a polymer film on the hair and fix the hair when used in a 0.01 to 5 percent by weight aqueous, alcoholic or aqueous-alcoholic solution or dispersion.

Suitable synthetic nonionic film-forming, hair-fixing polymers include homopolymers or copolymers, which are built up from at least one of the following monomers: vinyl pyrrolidone, vinyl caprolactam, vinyl esters, such as vinyl acetate, vinyl alcohols, acrylamides, methacrylamides, alkylacrylamides, dialkylacrylamides, alkylmethacrylamides, dialkylmethacrylamides, alkylacrylates, alkylmethacrylates, propylene glycol or ethylene glycol, wherein the alkyl groups of these monomers preferably are C₁- to C₇-alkyl groups, especially preferably C₁- to C₃-alkyl groups. For example, homopolymers of vinyl caprolactams, of vinyl pyrrolidone or of N-vinylformamide are suitable. Further suitable synthetic film-forming, non-ionic hair-fixing polymers are, e.g., copolymerizates of vinyl pyrrolidone and vinyl acetate, terpolymers of vinyl pyrrolidone, vinyl acetate and vinyl propionate, polyacrylamides, polyvinyl alcohols and polyethylene glycol/polypropylene glycol copolymers. Polyvinyl pyrrolidone and polyvinyl-pyrrolidone/vinyl acetate copolymers are especially preferred.

Suitable anionic hair-fixing polymers include synthetic homo-polymers or copolymers with monomer units containing acid groups, which are copolymerizable with comonomers, if necessary, which contain no acid groups. The acid groups may include —COOH, —SO₃H, —OSO₃H, —OPO₂H, —OPO₃H₂, of which carboxylic acid groups are particularly preferred. The acid groups can be unneutralized, or partially or completely neutralized. Preferably they are present in 50 to 100% anionic or neutralized form. Organic or inorganic bases suitable for cosmetic purposes can be used as neutralization agents. For example, suitable bases include aminoalcohols, such as aminomethylpropanol (AMP), triethanolamine, monoethanolamine or tetrahydroxypropylethyleneamine and ammonia, NaOH and others. Suitable monomers include unsaturated, radical polymerizable compounds, which have at least one acid group, especially carboxyvinyl monomers. Suitable monomers containing acid groups include e.g. acrylic acid, methacrylic acid, crotonic acid, maleic acid, maleic acid anhydride or their monoesters, aldehydocarboxylic acids or ketocarboxylic acids.

The comonomers not substituted with acid groups include, e.g., acryl amide, methacrylamide, alkyl- and dialkylacrylamides, alkyl- and dialkylmethacrylamides, alkylacrylates, alkylmethacrylates, vinyl caprolactone, vinyl pyrrolidone, vinyl esters, vinyl alcohol, propylene glycol or ethylene glycol, amine-substituted vinyl monomers, such as dialkylaminoalkylacrylates, dialkylaminoalkyl-methacrylates, monoalkylaminoalkylacrylates and monoalkylaminoalkyl-methacrylates, in which the alkyl groups of these monomers are preferably C₁- to C₇-alkyl groups, especially preferably C₁- to C₃-alkyl groups.

Suitable anionic polymers include especially copolymers of acrylic acid or methacrylic acid with monomers selected from the group consisting of acrylic acid or methacrylic acid esters, acryl amides, methacrylamides and vinyl pyrrolidones, homopolymers of crotonic acid and copolymers of crotonic acid with monomers selected from the group consisting of vinyl esters, acrylic acid esters, methacrylic acid esters, acrylamides and methacrylamides. A suitable natural polymer is, for example, shellac.

Preferred polymers with acid groups include cross-linked or uncross-linked vinyl acetate/crotonic acid copolymers. Preferred anionic polymers also include partially esterified copolymers of vinyl methyl ether and maleic acid anhydride. Other preferred anionic polymers include, for example, terpolymers of acrylic acid, alkyl acrylate and N-alkylacrylamide, especially acrylic acid/ethyl acrylate/N-t-butylacrylamide terpolymer or terpolymers of vinyl acetate, crotonate and vinyl alkanoate, especially vinyl acetate/crotonate/vinyl neodecanoate copolymers.

Suitable hair-fixing amphoteric polymers are polymers, which contain basic or cationic groups, especially primary, secondary, teriary or quaternary amine groups, besides acid or anionic groups. For example, amphoteric polymers can be copolymers formed from alkylacrylamides (especially octylacryl amide), alkylaminoalkylmethacrylate (especially t-butylaminoethylmethacrylate) and two or more monomers, namely acrylic acid, methacrylic acid or their esters, such as those marketed under the trademarks, AMPHOMER® or AMPHOMER® LV-71 of National Starch, USA. Further examples of hair-fixing copolymers with acid groups are copolymers of acrylic acid, methacrylate and methacrylamido-propyltrimethylammonium chloride (INCI: polyquaternium-47), which for example is marketed under the trademark® MERQUAT® 2001; copolymers made from acrylamidopropyltrimethylammonium chloride and acrylates or copolymers made from acrylamide, acrylamidopropyltrimethylammonium chloride, 2-amidopropyl-acrylamide sulfonate and dimethylaminopropylamine (DMAPA) (INCI: Poly-quaternium-43). Polymers with monomers carrying betaine groups are also suitable, such as copolymers of methacryloylethyl betaine and two or more monomers of acrylic acid or its simple esters, INCI name: Methacryloyl ethyl betaine/acrylates copolymer. Zwitterionic amphoteric polymers, which are described in JP 10-29919 or in JP 10-25344, are also suitable.

Suitable cationic polymers are polymers with cationic or basic, i.e. cationizable, groups. These polymers contain nitrogen-containing groups, such as primary, secondary or tertiary amines. These basic groups are either part of the polymer chain or preferably substituents on one or more of the monomers. The polymers with the basic groups can be natural or synthetic homo-polymers or copolymers with amine-substituted monomer units and, if necessary, with non-basic comonomers. Suitable polymers with basic groups are, e.g., copolymers of amine-substituted vinyl monomers and non-amine-substituted vinyl monomers. Amine-substituted vinyl monomers include dialkylaminoalkyl acrylates, dialkyl-aminoalkylmethacrylates, monoalkylaminoalkylacrylates and monoalkylamino-alkylmethacrylates, in which the alkyl groups of these monomers are preferably lower alkyl groups, such as alkyl groups with one to seven carbon atoms, especially preferably with from one to three carbon atoms.

For example, acrylamides, methacrylamides, alkyl- and dialkylacryl-amides, alkyl- and dialkylmethacrylamides, alkylacrylates, alkylmethacrylates, vinyl caprolactones, vinyl pyrrolidones, vinyl esters, vinyl alcohol, maleic acid anhydride, propylene glycol or ethylene glycol are especially suitable as non-amine-substituted comonomers. Comonomers in which the alkyl groups have one to seven carbon atoms, especially one to three carbon atoms, are particularly preferred.

Suitable cationic polymers with cationic groups preferably contain quaternary amine groups. The cationic polymers can be homo-polymers or copolymers, in which the quaternary nitrogen groups are either built into the polymer chain or preferably are contained as substituents on one or more monomers. The monomers containing the ammonium groups can be copolymerized with the above-described non-amine-substituted monomers. Suitable ammonium-substituted vinyl monomers include, e.g., trialkylmethacryl-oxyalkyl ammonium, trialkylacryloxyalkyl ammonium, dialkyldiallyl ammonium and quaternary vinyl ammonium monomers with cyclic, cationic nitrogen containing groups, such as pyridinium imidazolium or quaternary pyrrolidone, e.g. alkylvinylimidazolium alkylvinylpyridinium, or alkylvinylpyrrolidone salts. The alkyl groups of these monomers are preferably lower alkyl groups, in which the alkyl groups preferably have one to seven carbon atoms, especially one to three carbon atoms.

Suitable polymers with quaternary amine groups are, for example, the polymers described with the trade name “Polyquaternium” in the CTFA Cosmetic Ingredient Dictionary, such as methylvinylimidazolium chloride/vinylpyrrolidone copolymer (Polyquaternium-16), quaternized vinyl pyrrolidone/dimethylamino-ethylmethacrylate copolymer (Polyquaternium-11), homo-polymers and copolymers of dimethyldiallyl ammonium chloride (Polyquaternium-6 and -7), quaternarized hydroxyethylcellulose (Polyquaternium-10), quaternarized guar derivatives or polymers of vinyl pyrrolidone/dimethylaminopropylmethacrylamide and methacryloylaminopropyllauryldimethylammonium chloride (Polyquaternium-55, STYLEZE® W-20).

Polyvinylpyrrolidone/dimethylaminoethylmethacrylate copolymer is also a suitable cationic film-forming polymer, which can be contained in the composition according to the invention. Additional suitable cationic polymers include, e.g., the copolymer of polyvinyl pyrrolidone and imidazoliminemethochloride, the terpolymer of dimethyldiallylammonium chloride, sodium acrylate and acrylamide, the terpolymer of vinyl pyrrolidone, dimethylaminoethylmethacrylate and vinyl caprolactam, quaternary ammonium salts of hydroxyethyl cellulose and a trimethylammonium-substituted epoxide and vinyl pyrrolidone/methacrylamido-Propyltrimethyl ammonium chloride copolymers.

### Solid Particulate (E)

The hair treatment composition preferably contains from 0.1 to 5, especially 0.5 to 2, percent by weight of a solid particulate (E). The particulate can have different forms and densities. It can be spherical, oval or irregular. The average particle size, e.g., can be from 1 nm to 100 microns. The particle sizes, i.e. the maximum distance between two points on the particle, can, e.g., be determined with a laser granulometer.

Solid particulates are, for example, those based on talc, kaolin (hydrated aluminum silicate), precipitated carbonates, precipitated hydrogen carbonates, hydroxyapatite, silica, silicates, aluminates, alumina, mica; salts, especially inorganic metal salts; metal oxides, e.g. titanium dioxides, zinc oxides, aluminum oxides, zirconium oxides, cerium oxides; minerals and undissolved polymer particles that do not form a film on the hair, e.g. in the form of spherical microparticles (microspheres) or non-spherical microparticles, polymer powders, expanded microspheres, silicone microspheres, and polysaccharide microspheres.

Solid particulates include especially NYLON® powder, polyethylene powder, poly-beta-alanine powder, polyperfluoroalkylene powder, acryl copolymer powder, acrylate polymer powder, polyamido powder, polystyrene powder, polyester powder, polyurethane powder, expanded microspheres from thermoplastic materials, e.g. Expancel 551 DE, silicone resin micro particulates, e.g. tospearl. Silica is especially preferred, especially with an average particle size of from 1 to 100 nm or from 4 to 20 nm as well as pearlescent pigments with an average particle size of from 1 to 100 micrometers or from 10 to 60 micrometers.

Further preferred particles are pigment particles or paint particles, e.g. those selected from one or more organic pigments, inorganic-organic mixed pigments, inorganic pigments of natural origin, white pigments, black pigments, fluorescent pigments, phosphorescent pigments, colored metal oxides, colored metal hydroxides, colored metal oxide hydrates, colored mixed phase pigments, colored sulfur-containing silicates, colored metal sulfides, colored complex metal cyanides, colored metal sulfates, colored metal chromates, colored metal molybdates, bronze pigments, black iron oxide, yellow iron oxide, red iron oxide, brown iron oxide, manganese violet, ultramarine, chromium oxide hydrate, iron blue, carmine, and pigments based on mica or isinglass, which are coated with a metal oxide or metal oxychloride as well as a color-imparting material selected from the group consisting of iron oxides, iron blue, ultramarine, carmine and chromium oxide.

In an especially preferred embodiment of the hair treatment composition according to the invention the solid particulate is a pigment based on glass, e.g. borosilicate glass, preferably coated with metal oxides. The metal oxides can be for example iron oxides, titanium dioxide and/or tin oxides. Pigments based on glass with the INCI name calcium sodium borosilicate are examples of these pigments. Commercial products include, e.g., those with the trademark REFLECKS® or REFLECKS®) Dimensions, e.g. REFLECKS® Dimensions Sparkling Blue.

### Preferred Embodiment

A preferred embodiment of the hair treatment composition according to the invention contains:
(A) from 50 to 65 percent by weight water, based on a total amount of the composition;
(B) from 5 to 10 percent by weight of said at least one hydrophobic substance, based on a total amount of the composition, wherein the at least one hydrophobic substance is selected from the group consisting of fatty alcohols, vegetable oils, hydrocarbons that are liquid at room temperature and silicone oils;
(C) from 15 to 30 percent by weight of said at least one emulsifier, based on a total amount of the composition;
(D) from 0.1 to 15 percent by weight of at least one hair-fixing polymers, which is a non-ionic and/or cationic vinyl lactam polymer, especially polyvinyl pyrrolidone and/or Polyquaternium-55; and
(E) from 0.5 to 2 percent by weight, silica.

### Optional Cosmetic Ingredients

In addition to the above-named ingredients the product according to the invention can also contain additional conventional cosmetic additives:
cosmetic coloring materials in an amount of up to 6 percent by weight, preferably from 0.1 to 4 percent by weight, e.g. C.I. Pigment Red 4 (C.I. 12 085), C.I. Pigment Green (C.I. 74 260) and/or C.I. Vat Blue 4 (C.I. 69 800);
perfume and fragrance substances in an amount of up to 2 percent by weight, preferably from 0.01 to 1 percent by weight;
light-protective and preservative agents in an amount of up to 1 percent by weight, preferably from 0.01 to 0.5 percent by weight;
hair care additive ingredients, such as betaine, panthenol in an amount of up to 5 percent by weight, preferably from 0.01 to 4 percent by weight.

### Application and Packaging

The product according to the invention can be filled in a suitable package, e.g. a dish, tube, bottle or the like, according to its consistency. The package can be provided with a pumping device, e.g. a mechanically operated pump dispenser for applying the product mass.

A product for hair treatment, wherein a composition according to the invention is filled together with a suitable propellant in a pressurized container, which is provided with a device for producing foam (foam head), is a special embodiment of the invention. Suitable propellants are especially liquid propellant gas, such as propane, n-butane, isobutane, fluorinated hydrocarbons, such as 1,1-difluoroethane or 1,1,1,2-tetrafluoroethane or dimethyl ether. These propellant gases can be used individually or in mixtures with each other, e.g. a mixture of propane and/or butane and dimethylether. A mixture of propane and butane is especially preferred. Typical filling conditions for the pressurized container comprise about 80 to 98 percent by weight effective ingredient mixture to 2 to 20 percent by weight propellant. The pressurized container can be made from any known material suitable for aerosol spray or foam products. Suitable materials are especially metals, such as aluminum or tin plate. Commercial foam heads can be used as foam heads.

### Application Methods

A method of setting up a hair-do or hairstyle is part of the present invention. In the method according to the invention the hair treatment composition is applied to dry or moist hair and subsequently put in the desired hairstyle or hair-do. The product can be distributed on individual separated hair strands. The product preferably is worked into the separated hair strands from the hair roots to the tips. A pea-sized or hazelnut-sized amount is applied to the hair according to the hair length and desired effect. During the time, in which the product is still not completely dried on the hair, the strands may be easily put in the desired shape. For example, the hair strands can be simply twisted around themselves, starting from the hair roots, to form ropes. After complete drying the ropes are stabilized so that the hair has a pleasant feel without stickiness. The ropes can be treated again the following day with the composition according to the invention in order to maintain the hold. The ropes can be easily removed by simply washing the hair. The special advantages of the method are the short set up times and that the hair-do or hairstyle formed with the method is easily removable.

The following examples illustrate the subject matter of the invention in more detail without limitation of the appended claims.

### EXAMPLES

| Example 1 | LIQUID STYLER |
|---|---|
| 50-65 g | Water |
| 20 g | Emulsifier |
| 15 g | Hair-fixing polymers, e.g. mixture of Polyquaternium-55 |
| | and Polyvinyl pyrrolidone |
| 5 g | Polyhedric alcohol, e.g. propylene glycol |
| 5-10 g | Hydrophobic phase, e.g. fatty alcohol |
| 0.5-2 g | Silica |

| Example 2 | LIQUID STYLER |
|---|---|
| 3.4 g | PEG-25 Hydrogenated Castor Oil |
| 3.3 g | PEG-40 Hydrogenated Castor Oil |
| 3.3 g | Ceteareth-25 |
| 5 g | Polyquaternium-55 |
| 10 g | Polyvinyl pyrrolidone |
| 5 g | Polyhedric alcohol, e.g. propylene glycol |
| 7.50 g | Cetyl alcohol |
| 1.0 g | Silica |
| To 100 g | Water |

| Example 3 | AEROSOL FOAM |
|---|---|
| 50 g | Water |
| 25 g | Emulsifier |
| 10-15 g | Hair-fixing polymers, e.g. mixture of Polyquaternium-55 |
| | and Polyvinyl pyrrolidone |
| 3-6 g | Polyhedric alcohol, e.g. propylene glycol |
| 5-10 g | Hydrophobic phase, e.g. fatty alcohol |
| 0.5-2 g | Silica |
| 10 g | Propellant, e.g. propane, n-butane |

| Example 4 | AEROSOL FOAM |
|---|---|
| 50 g | Water |
| 25 g | Emulsifier |
| 6 g | Polyquaternium-55 |
| 6 g | Polyvinyl pyrrolidone |
| 4.5 g | Propylene glycol |
| 7.5 g | Cetyl alcohol |
| 1.0 g | Silica |
| 10 g | Propane/n-butane |

| Example 5 | COLOR-EFFECT LIQUID STYLER |
|---|---|
| 3.4 g | PEG-25 Hydrogenated Castor Oil |
| 3.3 g | PEG-40 Hydrogenated Castor Oil |
| 3.3 g | Ceteareth-25 |
| 5 g | Polyquaternium-55 |
| 10 g | Polyvinyl pyrrolidone |
| 5 g | Polyhedric alcohol, e.g. propylene glycol |
| 7.50 g | Cetyl alcohol |
| 1.0 g | REFLECKS ® Dimensions Sparkling Blue |
| | (INCl: Calcium Sodium Borosilicate (and) |
| | Titanium Dioxide (and) Tin Oxide) |
| To 100 g | Water |

The disclosure in German Patent Application P 04 001 875.6 of Jan. 29, 2004 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in an emulsion-form hair treatment composition containing solid particles, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

1. A hair treatment composition in the form of an emulsion, said emulsion comprising an aqueous hydrophilic phase and a hydrophobic phase, wherein said hair treatment composition comprises
at least one 50 percent by weight water, based on a total weight of the composition;
from 5 to 10 percent by weight of at least one hydrophobic substance, based on the total weight of the composition;
at least 15 percent by weight of at least one emulsifier, based on the total weight of the composition;
at least one hair-fixing polymer; and
at least one undissolved solid particulate.

2. The hair treatment composition as defined in claim 1, containing
from 50 to 65 percent by weight of said water;
from 5 to 10 percent by weight of said at least one hydrophobic substance;
from 15 to 30 percent by weight of said at least one emulsifier;
from 0.1 to 15 percent by weight of said at least one hair-fixing polymer; and
from 0.5 to 2 percent by weight of said at least one undissolved solid particulate.

3. The hair treatment composition as defined in claim 1, wherein said aqueous hydrophilic phase is a mixture of said water and one or more polyhedric C₁- to C₆-alcohol.

4. The hair treatment composition as defined in claim 1, wherein said at least one emulsifier is selected from the group consisting of
addition products of 2 to 30 mol ethylene oxide and/or 1 to 5 mol propylene oxide with C₈- to C₂₂-fatty alcohols, with C₁₂- to C₂₂-fatty acids or with alkylphenols containing 8 to 15 carbon atoms in the alkyl groups, C₁₂- to C₂₂-fatty acid monoesters and diesters of addition products of 1 to 30 mol ethylene oxide-with glycerol,
addition products of 5 to 60 mol of ethylene oxide with castor oil or hydrogenated castor oil, and
monoesters, diesters and triesters of phosphoric acid with addition products of 2 to 30 mol of ethylene oxide with C₈- to C₂₂-fatty alcohols;
or mixtures thereof.

5. The hair treatment composition as defined in claim 1, wherein said at least one hydrophobic substance is selected from the group consisting of fatty alcohols, vegetable oils, hydrocarbons that are liquid at room temperature and silicone oils.

6. The hair treatment composition as defined in claim 1, wherein said at least one hair-fixing polymer is selected from the group consisting of nonionic polymers, anionic polymers, cationic polymers and amphoteric polymers;
wherein said nonionic polymers each contain at least one monomer selected from the group consisting of vinyl lactams, vinyl esters, vinyl alcohols, acrylamides, methacrylamides, alkylacrylamides, dialkylacrylamides, alkylmethacrylamides, dialkylmethacrylamides, alkylacrylates, alkylmethacrylates, propylene glycol and ethylene glycol; and wherein alkyl groups in said nonionic polymers are C₁- to C₇-alkyl groups;
wherein said anionic polymers are built up from at least one monomer selected from the group consisting of acrylic acid, methacrylic acid, crotonic acid, maleic acid, maleic acid anhydride, monoesters of acrylic acid, monoesters of methacrylic acid, monoesters of crotonic acid, monoesters of maleic acid, aldehydocarboxylic acids and ketocarboxylic acids;
wherein said cationic polymers are built up from at least one monomer containing at least one cationic or cationizable group; and
wherein said amphoteric polymers are built up from at least one monomer, which contains both at least one acid group and at least one cationic or cationizable group or which contains at least one first monomer with said at least one acid group and at least one second monomer which contains said at least one cationic or cationizable group.

7. The hair treatment composition as defined in claim 1, wherein said at least one solid particulate comprises talc, polymer powders, expanded microspheres, silicone microspheres, precipitated carbonates, precipitated hydrogen carbonates, hydroxyapatite, silica, silicates, aluminates, alumina, mica, salts, metal oxides, polysaccharide microspheres and/or pigments based on borosilicate glass particulate coated with metal oxides.

8. The hair treatment composition as defined in claim 1, containing
from 50 to 65 percent by weight of said water;
from 5 to 10 percent by weight of said at least one hydrophobic substance, wherein said at least one hydrophobic substance is selected from the group consisting of fatty alcohols, vegetable oils, hydrocarbons that are liquid at room temperature and silicone oils;
from 15 to 30 percent by weight of said at least one emulsifier;
from 0.1 to 15 percent by weight of at least one non-ionic and/or cationic vinyl lactam polymer; and
from 0.5 to 2 percent by weight of silica.

9. The hair treatment composition as defined in claim 1, further comprising at least one propellant.

10. A method of setting up a hairstyle or hair-do, said method comprising the steps of:
a) applying a hair treatment composition as defined in claim 1 to dry or moist hair; and
b) subsequently putting the hair in a hairstyle or hair-do.
